(19) United States
(12) Patent Application Publication
(10) Pub. No.: US 2021/0052437 A1
(43) Pub. Date: Feb. 25, 2021

(54) FLEXIBLE SANITARY PAD FITTED TO THE GROIN

(71) Applicant: Ching-Ju Wu, Kaohsiung City (TW)

(72) Inventor: Ching-Ju Wu, Kaohsiung City (TW)

(21) Appl. No.: 16/553,618

(22) Filed: Aug. 28, 2019

(30) Foreign Application Priority Data
Aug. 22, 2019 (CN) PCT/CN2019/102090

(51) Int. Cl. A61F 13/475; A61F 13/53

(52) U.S. Cl. CPC A61F 13/4753; A61F 2013/4512; A61F 13/53

(57) ABSTRACT

Provided is a flexible sanitary pad to solve the problems of leakage of menstrual blood of the conventional sanitary pad. The sanitary pad of the present invention includes an absorbent pad and a fluid guide. The fluid guide has a suspended portion positioned between a front fixed portion and a back fixed portion. The fluid guide also has and a tilted tail portion. The front fixed portion and the back fixed portion connect to the absorbent pad. The tilted tail portion is positioned at a back end of the back fixed portion. A head end of the tilted tail portion is more adjacent to the absorbent pad than a tail end of the tilted tail portion. The tail end of the tilted tail portion is suspended without touching the absorbent pad.

### CROSS REFERENCE TO RELATED APPLICATION

The application claims the benefit of P.C.T International Patent Application Serial No. PCT/CN2019/102090, filed on Aug. 22, 2019, and the entire contents of which are incorporated herein by reference.

### BACKGROUND OF THE INVENTION

### 1. Field of the Invention

The present invention relates to a feminine hygiene product and, more particularly, to a flexible sanitary pad fitted to the groin for absorbing the menstrual blood.

### 2. Description of the Related Art

Generally speaking, two kinds of the sanitary pad for day use and night use may be chosen according to the timing of use. Less menstrual blood flowing back along the groin is happened in the daytime because female mostly stands or sits down in the daytime. Thus, the length between 23 to 26 cm is enough for the sanitary pad for day use. Conversely, part of the menstrual blood may flow back along the groin if the female sleeps on her back at night. Thus, the length of the sanitary pad for night use may be elongated with common length of between 28 to 35 cm. Few sanitary pads on the market have lengths longer than 43 cm.

In addition, a commodity named “menstrual pants” is sold on the market. The shape of the menstrual pants is similar to that of common pants, and a fiber layer easily to be decontaminated is disposed on an inner layer of the menstrual pants. A user with less menstrual blood may merely wear the menstrual pants, allowing the menstrual blood directly to be absorbed by the menstrual pants. A user with more menstrual blood may wear the menstrual pants with a sanitary article such as a sanitary pad or a tampon, allowing the leakage of menstrual blood to be absorbed by the menstrual pants. However, leaking of menstrual blood exceeded the absorption range still happens due to the fiber layer for absorbing the menstrual blood occupied only part of the inner layer. In addition, although wearing the menstrual pants along with the sanitary article decreases the chance of leaking the menstrual blood, it may also cause stuffy feeling.

For the above mentioned commodities, the reason that the leakage of menstrual blood could not be efficiently prevented even using the longer sanitary pad or more protection layers is that the sanitary pad or the menstrual pants with a plane type may not extend into the groin of the user. Thus, as the user sleeps on her back, part of the menstrual blood still may flow back along the groin and exceed the absorption region, or lateral leakage of menstrual blood may happen due to turning of the user's body. Furthermore, higher cost and price of the elongated sanitary pads and the menstrual pants unable to prevent menstrual blood from leaking may cause troubles and financial burden of the user.

Thus, a conventional sanitary pad is necessary to be improved.

### SUMMARY OF THE INVENTION

To solve the problems mentioned above, the purpose of the present invention is to provide a flexible sanitary pad fitted to the groin, which may extend into and fit to the groin, allowing the menstrual blood flowed to be absorbed immediately and guided to a correct position, thereby efficiently decreasing the chance of leaking the menstrual blood.

Another purpose of the present invention is to provide a flexible sanitary pad fitted to the groin with a moderate length without being elongated. The flexible sanitary pad does not need to be used together with the menstrual pants. Such feature may also efficiently decrease the chance of leaking the menstrual blood, the financial burden of the user and stuffy feeling in use.

Another purpose of the present invention is to provide a flexible sanitary pad fitted to the groin, which may efficiently prevent the menstrual blood from leaking forward as the user sleeps on her stomach or leans forward.

“Front” and “back” recited in the disclosure is described according to the direction as using the sanitary pad. In addition, “front” refers to the direction toward the “ventral side”, “back” refers to the direction toward the “dorsal”. “Front” and “back” described in the disclosure are merely relative relationship, rather than referring to “anterior” or “posterior” defined in human anatomy based on the frontal plane. Other directionality or similar terms, such as “left”, “right”, “up (top)”, “down (bottom)”, “inner”, “outer” and “lateral side” are mainly referred to the direction of drawings. Each directionality or similar terms have reference only to the structure shown in the drawings as it would appear to a person viewing the drawings, and are utilized only to facilitate describing the invention, rather than limiting the invention.

“A” or “an” used in the elements and components recited in the disclosure of the present invention is for convenient use and provides generally meanings of the scope of the present invention. “A” or “an” used in the present invention is interpreted as comprising one or at least one, and the single concept also includes plural conditions, otherwise obviously indicating other meanings.

“Connection”, “combination” or “assembling” and similar terms described in the full text of the present invention mainly include types of the components detached without destroying the components after connecting, or components which are inseparable after connecting, which may be chose by a person ordinarily skilled in the art according to the component material for assembling or assembly requirements.

A flexible sanitary pad fitted to the groin of the present invention includes an absorbent pad and a fluid guide. The fluid guide has a suspended portion and a tilted tail portion. The suspended portion is positioned between a front fixed portion and a back fixed portion. The front fixed portion and the back fixed portion connect to the absorbent pad. The tilted tail portion is positioned at a back end of the back fixed portion, and a head end of the tilted tail portion is more adjacent to the absorbent pad than a tail end of the tilted tail portion. The tail end of the tilted tail portion is suspended without touching the absorbent pad.

Accordingly, a flexible sanitary pad fitted to the groin of the present invention allows the menstrual blood to be guided to the absorbent pad and absorbed immediately as the menstrual blood outflows by the suspended portion of the fluid guide fitted to the vulva and groin of the user. The tilted tail portion of the fluid guide extended into and fitted to the groin of the user allows the menstrual blood flowing back along the groin to be absorbed by the tilted tail portion. Thus, the sanitary pad of the present invention may efficiently decrease the chance of leaking menstrual blood and increase the sense of security of the user, allowing the action or sleeping posture not to be limited during the menstrual period. The present invention may absorb and guide the menstrual blood to the absorbent pad by the fluid guide, allowing the length of whole sanitary pad to be moderate without being elongated. Furthermore, the present invention may efficiently decrease the chance of leaking menstrual blood without wearing the menstrual pants. As such, the present invention may decrease the financial burden of the user and stuffy feeling in use, and solve the problem of frequent cleaning of clothes and bedclothes during the menstrual period.

In an example, the fluid guide may be exposed on an upper surface of the absorbent pad. As such, it may enhance the absorbing effect and guiding efficiencies.

In an example, the tail end of the tilted tail portion may be higher than the suspended portion. As such, the tilted tail portion may roughly align with the user's groin for absorbing the menstrual blood flowing back along the groin. It may provide the effect of prevention of leakage of the menstrual blood.

In an example, the suspended portion has a main absorbent segment. A head end of the main absorbent segment may be more adjacent to the absorbent pad than a tail end of the main absorbent segment. The head end of the main absorbent segment may connect to the front fixed portion. The tail end of the main absorbent segment may connect to the back fixed portion by a connection portion, and the tail end of the main absorbent segment may be positioned at the back end of the back fixed portion. As such, the main absorbent segment adjacent to the tail end may be flexible, allowing the flexible absorbent segment adjacent to the tail to be fitted to the vulva or groin of the user after being pressed. It may provide the effect of prevention of leakage of the menstrual blood.

In an example, the head end of the tilted tail portion may connect to the back fixed portion. The tail end of the main absorbent segment may be aligned with a region between the back fixed portion and the tail end of the tilted tail portion. A length of the tilted tail portion may be longer than the connection segment. As such, the tilted tail portion may be moved along with the suspended portion, allowing the tilted tail portion to be tilted up as pressing the suspended portion. It may provide the effect of prevention of leakage of the menstrual blood.

In an example, the suspended portion may have a main absorbent segment. A head end of the main absorbent segment may be the front fixed portion, and a tail end of the main absorbent segment may be the back fixed portion. As such, the structure of the fluid guide may be simplified. It may provide the effect of enhancing manufacturing convenience.

In an example, the suspended portion has a main absorbent segment. A head end of the main absorbent segment may connect to the back fixed portion by a connection portion, and the head end of the main absorbent segment may be positioned at a front end of the front fixed portion. A tail end of the main absorbent segment connects to the back fixed portion by another connection segment, and the tail end of the main absorbent segment is positioned at a back end of the back fixed portion. As such, the main absorbent segment adjacent to the head end and the tail end may be flexible, allowing the flexible absorbent segment adjacent to the head end and the tail end to be fitted to the vulva or groin of the user after being pressed. It may provide the effect of prevention of leakage of the menstrual blood.

In an example, the main absorbent segment may be tilted forward. As such, it may meet the requirement of ergonomics and provide the effect of enhancing comfortability.

In an example, the fluid guide may have a plurality of bended guide portion positioned between the head end of the main absorbent portion and the tail end of the main absorbent portion. A thickness of the fluid guide at the bended guide portion is smaller than a thickness of the fluid guide not at the bended guide portion. As such, the main absorbent segment may be deformed in a desired shape, thereby allowing enough desired part of the main absorbent segment to be attached to the absorbent pad to guide the menstrual blood to be absorbed uniformly. It may provide the effect of prevention of leakage of the menstrual blood.

In an example, the fluid guide may have a front guide portion positioned at a front end of the front fixed portion. A head end of the front guide portion may be suspended without touching the absorbent pad, and the head end of the front guide portion may be tilted up. As such, the fluid guide may attach to the vulva of the user more tightly. It may provide the effect of prevention of leakage of the menstrual blood.

In an example, the head end of the front guide portion may be higher than the suspended portion. As such, the menstrual blood flowing forward may be absorbed by the front guide portion, thereby preventing the menstrual blood from leaking forward as the user sleeps on her stomach or leans forward. It allows the user to feel free without limiting the activity or sleeping posture during the menstrual period.

In an example, the fluid guide may have a front guide portion positioned at a front end of the front fixed portion. The fluid guide may have a plurality of bended guide portion positioned between the head end of the main absorbent portion and the tail end of the main absorbent portion. A thickness of the fluid guide at the bended guide portion is smaller than a thickness of the fluid guide not at the bended guide portion. As such, it may ensure the front guide portion to be deformed in a desired shape, thereby allowing enough desired part of the front guide portion to be attached to the absorbent pad to guide the menstrual blood to be absorbed uniformly. It may provide the effect of prevention of leakage of the menstrual blood.

In an example, the fluid guide may have a back portion connected to the absorbent pad and abutted against the tilted tail portion. As such, the back portion may assist in maintaining the tilted tail portion to be tilted up and fitted to the groin. It may provide the effect of prevention of leakage of the menstrual blood.

In an example, the fluid guide may have an auxiliary block portion with a continuation segment connected to a abutting portion. The continuation segment may connect to the tail end of the tilted tail portion. A head end of the abutting portion may connect to the continuation segment, and a tail end of the abutting portion may be tilted up. As such, the auxiliary block portion may also be attached to the groin of the user, allowing the auxiliary block portion to absorb the menstrual blood flowed backward along the groin and not absorbed by the tilted tail portion. It may form multiple protection layers and provide the effect of prevention of leakage of the menstrual blood.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a stereoscopic exploded view of a flexible sanitary pad fitted to the groin according to the first embodiment of the present invention.

FIG. 2 is a stereoscopic assembled view of a flexible sanitary pad fitted to the groin according to the first embodiment of the present invention.

FIG. 3 is a side view of a flexible sanitary pad fitted to the groin according to the first embodiment of the present invention.

FIG. 4 is a cross sectional view along line A-A of FIG. 3.

FIG. 5 is a diagrammatic view showing the relative position of a flexible sanitary pad fitted to the groin and the breech of the user according to the first embodiment of the present invention.

FIG. 6 is a cross sectional view along line B-B of FIG. 5.

FIG. 7 is a side view of a flexible sanitary pad fitted to the groin according to the second embodiment of the present invention.

FIG. 8 is a side view of a flexible sanitary pad fitted to the groin according to the third embodiment of the present invention.

FIG. 9 is a side view of a flexible sanitary pad fitted to the groin according to the fourth embodiment of the present invention.

FIG. 10 is a side view of a flexible sanitary pad fitted to the groin according to the fifth embodiment of the present invention.

FIG. 11 is a side view of a flexible sanitary pad fitted to the groin according to the sixth embodiment of the present invention.

FIG. 12 is a side view of a flexible sanitary pad fitted to the groin according to the seventh embodiment of the present invention.

### DETAILED DESCRIPTION OF THE INVENTION

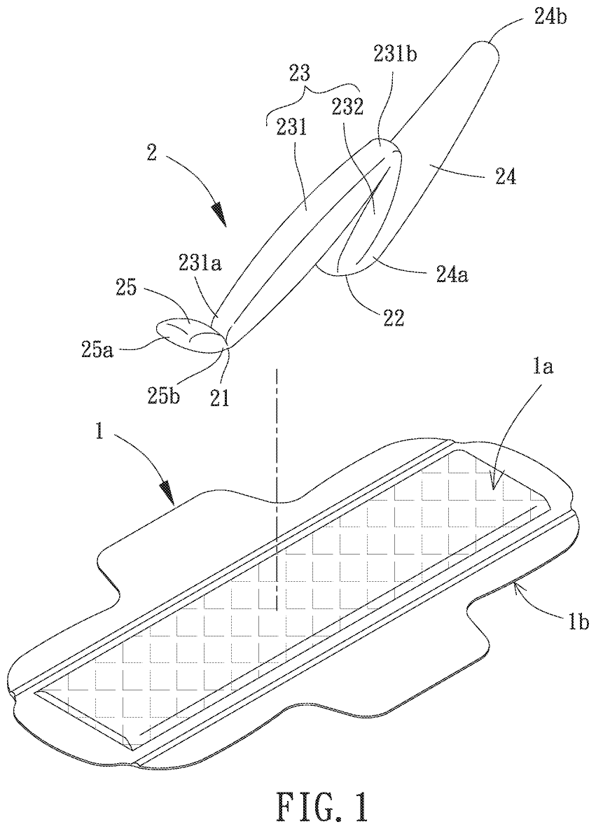

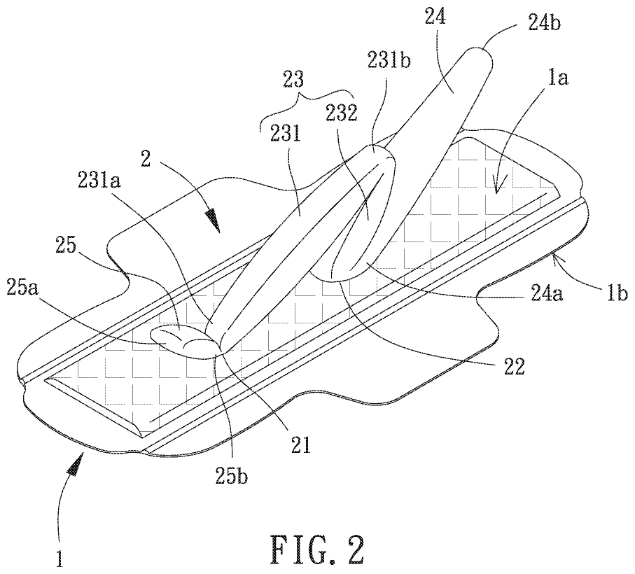

Referring to FIGS. 1 and 2, it is a flexible sanitary pad fitted to the groin of the first embodiment of the present invention, which includes an absorbent pad 1 and a fluid guide 2 connected to the absorbent pad 1.

The absorbent pad 1 may be any sanitary pad in any shape, and is not limited to the shape shown in the drawings of the present embodiment. That is, the absorbent pad 1 has an upper surface 1a and a lower surface 1b which is impervious. The function and shape of the remaining regions, as well as the materials used in the absorbent pad 1 for absorbing the menstrual blood are not limited in the present invention.

Referring to FIGS. 1 and 4, the fluid guide 2 is positioned on the upper surface 1a of the absorbent pad 1 for fitting into the vulva and groin of the user. It allows the menstrual blood outflowed from the vagina to be absorbed immediately and guided to the absorbent pad 1. It may prevent the menstrual blood from leaking from the sanitary pad and causing user inconvenience. In the present embodiment, for example, the fluid guide 2 may be formed in a long strip shape by filling with the high fluffy cotton C in an absorption net N. The fluid guide 2 may be exposed on the upper surface 1a of the absorbent pad 1, and connected to the absorbent pad 1 with a front fixed portion 21 and a back fixed portion 22. The front fixed portion 21 and the back fixed portion 22 may connect to the absorbent pad 1 by methods such as, but not limited to, adhesive, high frequency welding or stitching.

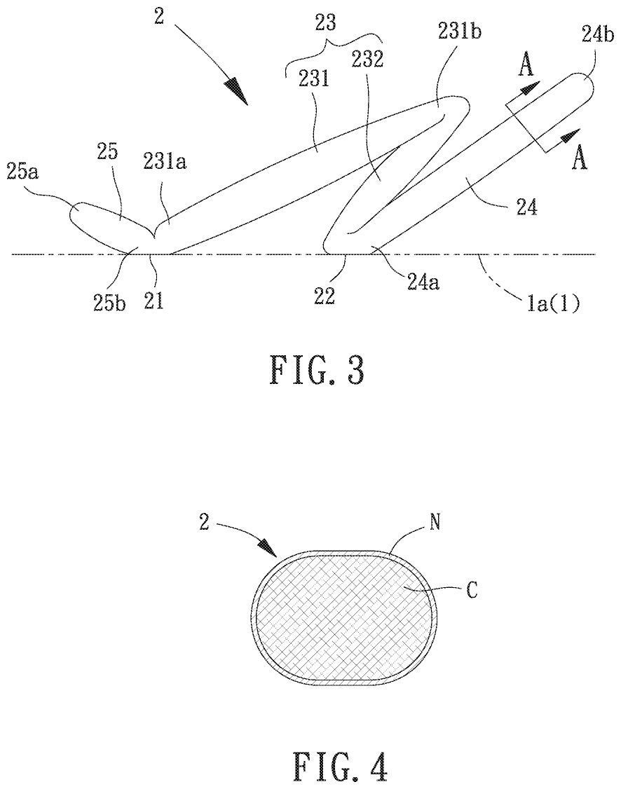

Referring to FIGS. 1 and 3, the fluid guide 2 has a suspended portion 23 positioned between the front fixed portion 21 and the back fixed portion 22. In principle, the suspended portion 23 does not touch the absorbent pad 1 without being pressed. As such, the upper surface 1a of the absorbent pad 1 protrudes enough to fit to the user's skin, and the region below the suspended portion 23 may form a breathable space because the suspended portion 23 does not attach to the absorbent pad 1 entirely. The suspended portion 23 has a main absorbent segment 231, and the vaginal ostium may roughly align with the main absorbent segment 231, allowing the menstrual blood to be absorbed immediately by the main absorbent segment 231. In the present embodiment, a head end 231a of the main absorbent segment 231 is more adjacent to the absorbent pad 1 than a tail end 231b of the main absorbent segment 231, allowing the main absorbent segment 231 to be in a tilted form. In addition, the head end 231a of the main absorbent segment 231 may directly or indirectly connect to the front fixed portion 21. The tail end 231b of the main absorbent segment 231 may directly or indirectly connect to the back fixed portion 22 by a continuation segment 232. The tail end 231b of the main absorbent segment 231 is positioned at a back end of the back fixed portion 22. A bended area is formed by the main absorbent segment 231 and the continuation segment 232, allowing the main absorbent segment 231 adjacent to the tail end 231b to be flexible and fitted to the vagina or groin of the user when being pressed. A length of the main absorbent segment 231 is preferably longer than that of the continuation segment 232.

The fluid guide 2 further has a tilted tail portion 24 positioned at a back end of the back fixed portion 22. A head end 24a of the tilted tail portion 24 is more adjacent to the absorbent pad 1 than a tail end 24b of the tilted tail portion 24, allowing the tilted tail portion 24 to be in a tilted form. The tail end 24b of the tilted tail portion 24 is suspended without touching the absorbent pad 1. The tail end 24b of the tilted tail portion 24 is preferably higher than the suspended portion 23, allowing the tilted tail portion 24 to be roughly aligned with the groin of the user and able to immediately absorb the menstrual blood flowed back along the groin. In the present embodiment, the head end 24a of the tilted tail portion 24 may directly or indirectly connect to the back fixed portion 22, allowing the tail end 231b of the main absorbent segment 231 of the suspended portion 23 to be roughly aligned with a region between the back fixed portion 22 and the tail end 24b of the tilted tail portion 24. The length of the tilted tail portion 24 is longer than that of the continuation segment 232, allowing the tilted tail portion 24 to be moved along the suspended portion 23. The tilted tail portion 24 may be tilted up more when the suspended portion 23 is pressed.

In addition, the fluid guide 2 may have a front guide portion 25 positioned at a front end of the front fixed portion 21. For example, but not limited to, the front guide portion 25 of the present embodiment may be in a form of single segment with a short length. A tail end 25b of the front guide portion 25 may directly or indirectly connect to the front fixed portion 21, allowing a head end 25a of the front guide portion 25 to be tilted up naturally and more fitted to the vulva of the user.

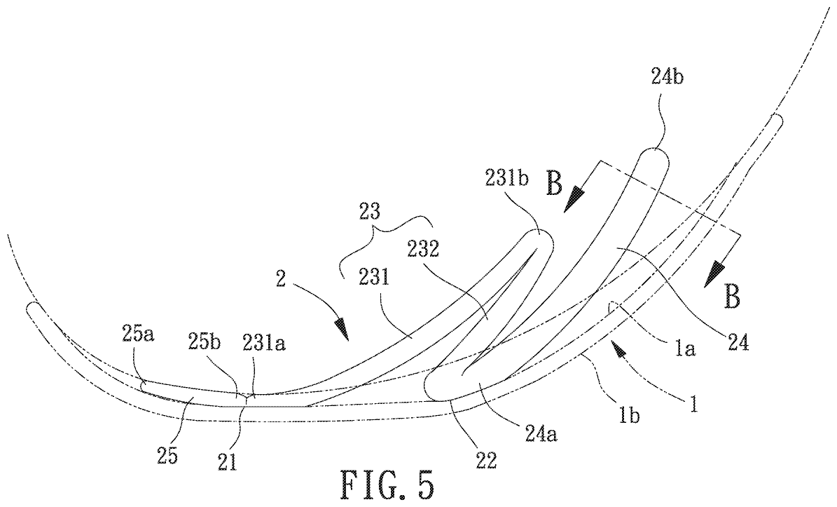

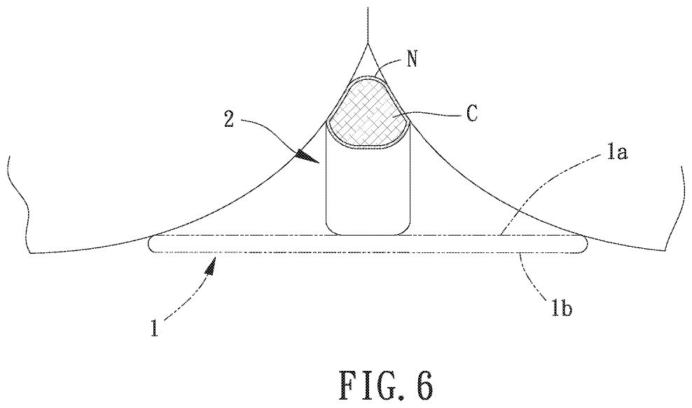

Referring to FIGS. 5 and 6, according to the structure mentioned above, as the absorbent pad 1 of the sanitary pad of the present embodiment is attached to the user's underwear, the fluid guide 2 may be in a predetermined stereoscopic form. When the user wears the underwear, the front guide portion 25 and the suspended portion 23 of the fluid guide 2 may elastically fit to the vulva of the user, and the suspended portion 23 and the tilted tail portion 24 of the fluid guide 2 may elastically fit to the groin of the user. As such, no matter what posture the user is in, the menstrual blood outflowed from the vaginal ostium may be absorbed immediately by the suspended portion 23. In addition, part of the suspended portion 23 (usually referring to the anterior part in the present embodiment) may attach to the absorbent pad 1 when the suspended portion 23 is pressed, thereby guiding the menstrual blood to the absorbent pad 1. Meanwhile, the tilted tail portion 24 may be more tilted up as pressing the suspended portion 23 and extended into and elastically fitted to the user's groin. Thus, even there is less time to absorb the huge amount of the menstrual blood by the suspended portion 23 as the user sleeps on her back, the menstrual blood flowed backward may also be absorbed by the tilted tail portion 24. Thus, it may decrease the chance of the leakage of the menstrual blood. In addition, part of the suspended portion 23 (usually referring to the posterior part in the present embodiment) has better breathability because the suspended portion 23 does not attach to the absorbent pad 1. It may decrease the stuffy feeling and enhance the comfortability as using the flexible sanitary pad.

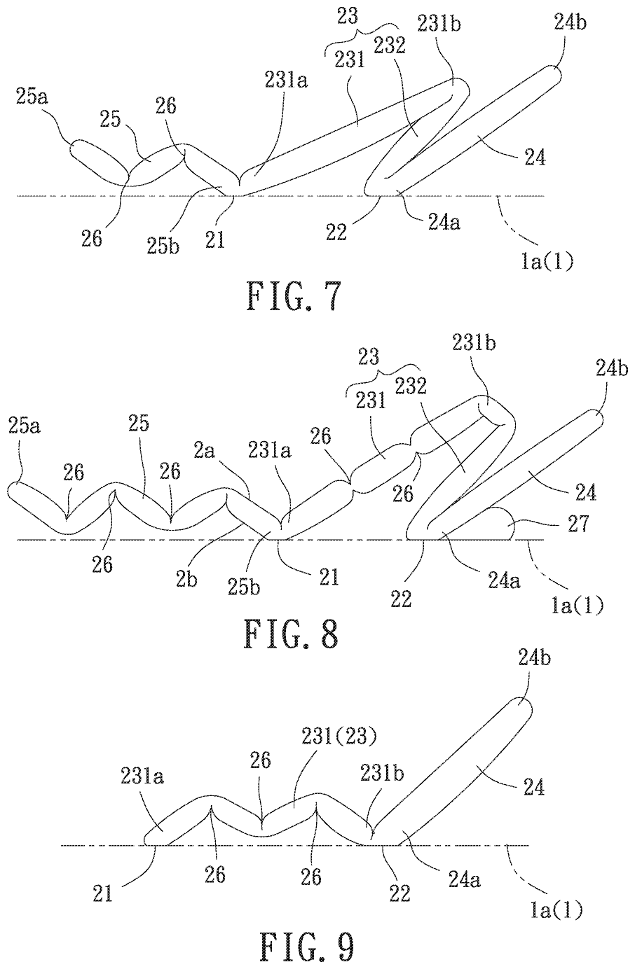

Referring to FIG. 7, it is a flexible sanitary pad fitted to the groin of the second embodiment of the present invention. The present embodiment is basically similar to the aforementioned first embodiment. In the present embodiment, a longer length of the fluid guide 2 may be remained to form the front guide portion 25. In addition, the fluid guide 2 has a plurality of bended guide portion 26 positioned between the head end 25a and the tail end 26a of the front guide portion 25, allowing a thickness of the fluid guide 2 at the bended guide portion 26 to be smaller than that of the fluid guide 2 not at the bended guide portion 26. The front guide portion 25 may form in a wavy shape extended forward based on the plurality of the bended guide portion 26. For example, the bended guide portion 26 may be thermoformed. As such, it is ensured that the front guide portion 25 may be deformed in a desired shape by the plurality of the bended guide portion 26, thereby allowing enough desired part of the main absorbent segment 231 to be attached to the absorbent pad 1 to guide the menstrual blood to be absorbed uniformly. It may provide the effect of prevention of leakage of the menstrual blood.

Referring to FIG. 8, it is a flexible sanitary pad fitted to the groin of the third embodiment of the present invention. The present embodiment is basically similar to the aforementioned second embodiment. In the present embodiment, aside from forming the front guide portion 25 in a wavy shape extended forward, a plurality of bended guide portion 26 may also be formed between the head end 231a and the tail end 231b of the main absorbent segment 231 of the suspended portion 23, allowing the main absorbent segment 231 to be formed into a shape with segments. In addition, the fluid guide 2 of the present embodiment may also have a back portion 27 connected to the absorbent pad 1 and abutted against the tilted tail portion 24. As such, the back portion 27 may assist in maintaining the tilted tail portion 24 to be tilted up and fitted to the groin. It may provide the effect of prevention of leakage of the menstrual blood.

Referring to FIG. 9, it is a flexible sanitary pad fitted to the groin of the fourth embodiment of the present invention. In the present embodiment, the head end 231a of the main absorbent segment 231 of the suspended portion 23 may act as the front fixed portion 21, and the tail end 231b of the main absorbent segment 231 may act as the back fixed portion 22, allowing the structure of the fluid guide 2 to be simplified and easy to be manufactured. More preferably, a plurality of bended guide portion 26 may also be deposed between the head end 231a and the tail end 231b of the main absorbent segment 231, allowing the main absorbent segment 231 to be formed into a shape with segments.

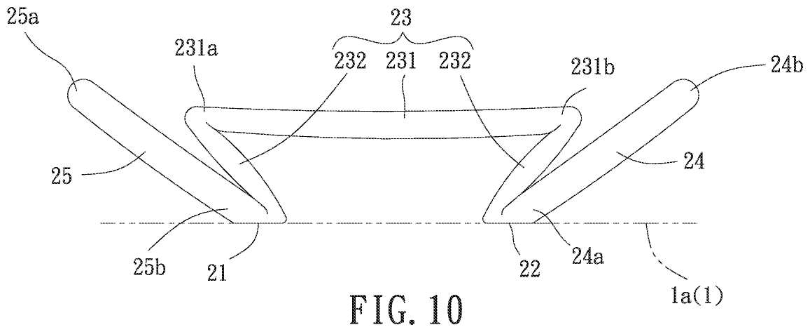

Referring to FIG. 10, it is a flexible sanitary pad fitted to the groin of the third embodiment of the present invention. The present embodiment is basically similar to the aforementioned first embodiment. In the present embodiment, the head end 231a of the main absorbent segment 231 of the suspended portion 23 may directly or indirectly connects to the front fixed portion 21 by a continuation segment 232, and the head end 231a of the main absorbent segment 231 is positioned at a front end of the front fixed portion 21, allowing the main absorbent segment 231 adjacent to the head end 231a to also be flexible and fitted to the vagina of the user when being pressed. It may enhance the interactions among the suspended portion 23, the front guide portion 25 and the tilted tail portion 24. For example, the clamping region between the head end 231a of the main absorbent segment 231 and the adjacent segment 232 is tighter when the front portion of the main absorbent segment 231 is pressed, allowing the clamping region between the tail end 231b of the main absorbent segment 231 and the adjacent segment 232 to be looser and tilted up, driving the tilted tail portion 24 to tilt up more and be more fitted to the groin. By contrast, the clamping region between the tail end 231b of the main absorbent segment 231 and the adjacent segment 232 is tighter when the back portion of the main absorbent segment 231 is pressed. The clamping region between the head end 231a of the main absorbent segment 231 and the adjacent segment 232 is looser and tilted up, driving the front guide portion 25 to tilt up more and be more fitted to the front of vulva.

In another aspect, the head end 25a of the front guide portion 25 may be suspended without touching the absorbent pad 1, and the head end 25a of the front guide portion 25 may be higher than the suspended portion 23, allowing the menstrual blood flowed forward to be absorbed by the front guide portion 25 as the user sleeps on her stomach. It allows the menstrual blood to be guide to the absorbent pad 1 by touching the absorbent pad 1 and the front fixed portion 21 after being pressed, thereby efficiently preventing the menstrual blood from leaking as the user sleeps on her stomach or leans forward. It allows the action or sleeping posture not to be limited during the menstrual period.

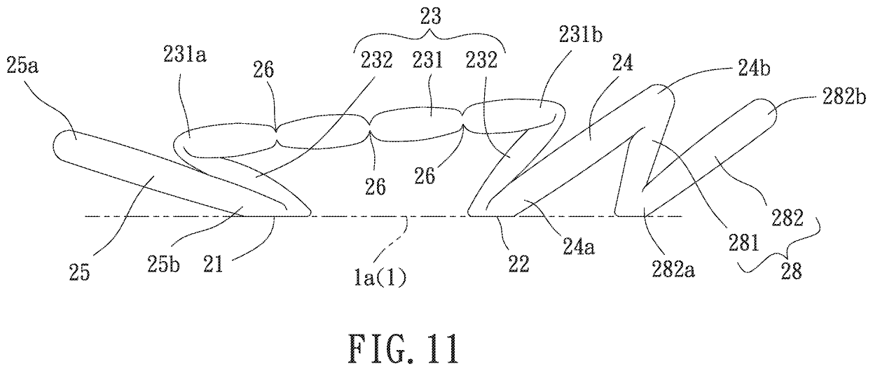

Referring to FIG. 11, it is a flexible sanitary pad fitted to the groin of the sixth embodiment of the present invention. The present embodiment is basically similar to the aforementioned fifth embodiment. In the present embodiment, the front fixed portion 21 is closer to the back fixed portion 22, or the segment 232 connected to the head end 231a of the main absorbent segment 231 is shorter. Such arrangement allows the main absorbent segment 231 of the suspended portion 23 to slightly inclined forward to meet the requirement of ergonomics and enhance comfortability. In addition, the fluid guide 2 may have a plurality of bended guide portion 26 positioned between the head end 231a and the tail end 231b of the main absorbent segment 231, allowing the main absorbent segment 231 to be formed into a shape with segments.

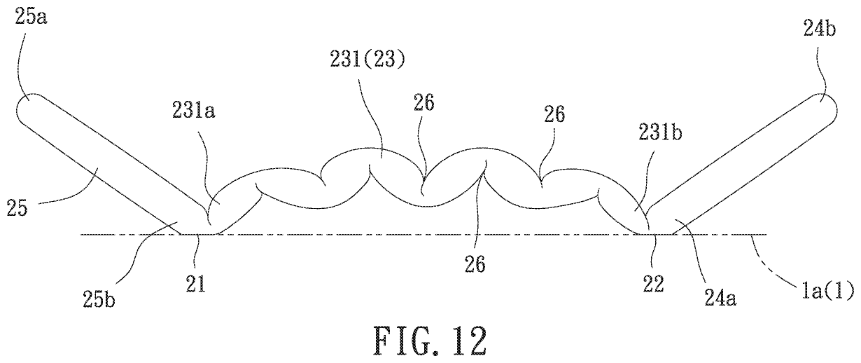

Referring to FIG. 12, it is a flexible sanitary pad fitted to the groin of the seventh embodiment of the present invention. The present embodiment is basically similar to the aforementioned fourth embodiment. In the present embodiment, the head end 231a of the main absorbent segment 231 of the suspended portion 23 may also connect to a front guide portion 25 in addition to acting as the front fixed portion 21. The head end 25a of the front guide portion 25 may be suspended without touching the absorbent pad 1. The head end 25 of the front guide portion 25 may be higher than the suspended portion 23 and have the same length with the tilted tail portion 24, allowing the menstrual blood flowed forward to be absorbed by the front guide portion 25.

In addition, the fluid guide 2 may also have at least one auxiliary block portion 28 positioned at the back end of the tilted tail portion 24. In detail, the auxiliary block portion 28 has a continuation segment 281 and an abutting portion 282. The continuation segment 281 may connect to the tilted tail portion 24, and the head end 282a of the abutting portion 282 connects to the continuation segment 281. Preferably, the head end 282a may also connect to the absorbent pad 1, and the tail end 282b of the abutting portion 282 is tilted at the back end of the tilted tail portion 24 to attach and fit to the groin of the user, allowing the menstrual blood flowed along the groin of the user that is not absorbed by the tilted tail portion 24 to be absorbed by the auxiliary block portion 28. It forms more layers of protection and provide the effect of preventing the menstrual blood from leaking. When more than one auxiliary block portions 28 are formed, the continuation segment 281 of the auxiliary block portion 28 will connect to the tail end 282b of a previous abutting portion 252, and sequentially connecting in series.

It is worth mentioning that any structure that may be implemented, such as the auxiliary block portion 28 or the plurality of the bended guide portion 26, may be used in any one of the embodiments mentioned above, and is not limited by the examples shown in the drawings. In addition, referring to FIG. 8, the embodiments mentioned above the fluid guide 2 may have an upper surface 2a and a lower surface 2b opposite to the upper surface 2a, with the lower surface 2b facing the absorbent pad 1. A distance between the upper surface 2a and the lower surface 2b is the thickness of the fluid guide 2. The bended guide portion 26 positioned on the upper surface 2a and the bended guide portion 26 positioned on the lower surface 2b may position in a misalignment pattern (such as shown at the front guide portion 25) or in an opposite position (such as shown at the main absorbent segment 231). That is, such alignment is not limited in the present invention.

In summary, the flexible sanitary pad fitted to the groin of the present invention may allow the outflowed menstrual blood to be immediately absorbed and guided to the absorbent pad by the suspended portion of the fluid guide fitted to the vulva and groin of the user. In addition, the tilted tail portion of the fluid guide extended into and fitted to the groin of the user allows the menstrual blood flowing backward along the groin to be absorbed by the tilted tail portion. Thus, the sanitary pad of the present invention may efficiently decrease the chance of leaking menstrual blood and increase the sense of security of the user, allowing the action or sleeping posture not to be limited during the menstrual period. Furthermore, the suspended portion has better breathability because the suspended portion does not attach to the absorbent pad, and it may decrease the stuffy feeling in use and enhance the comfortability.

In another aspect, the flexible sanitary pad fitted to the groin of the present invention may allow the menstrual blood to be absorbed by the guide portion and guided to the absorbent pad, allowing the length of the whole sanitary pad to be moderate without being elongated. The flexible sanitary pad fitted to the groin of the present invention may decrease the chance of leaking the menstrual blood and eliminate the needs of wearing the menstrual pants. As such, the present invention may decrease the financial burden of the user and stuffy feeling in use, enhance the comfortability, and solve the problem of frequent cleaning of clothes and bedclothes during the menstrual period.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A flexible sanitary pad fitted to the groin, comprising:
an absorbent pad; and
a fluid guide having a suspended portion positioned between a front fixed portion and a back fixed portion, wherein the fluid guide further has a tilted tail portion, wherein the front fixed portion and the back fixed portion connect to the absorbent pad, wherein the tilted tail portion is positioned at a back end of the back fixed portion, wherein a head end of the tilted tail portion is more adjacent to the absorbent pad than a tail end of the tilted tail portion, and wherein the tail end of the tilted tail portion is suspended without touching the absorbent pad.

2. The flexible sanitary pad fitted to the groin as claimed in claim 1, wherein the fluid guide is exposed on an upper surface of the absorbent pad.

3. The flexible sanitary pad fitted to the groin as claimed in claim 1, wherein the tail end of the tilted tail portion is higher than the suspended portion.

4. The flexible sanitary pad fitted to the groin as claimed in claim 1, wherein the suspended portion has a main absorbent segment, wherein a head end of the main absorbent segment is more adjacent to the absorbent pad than a tail end of the main absorbent segment, wherein the head end of the main absorbent segment connects the front fixed portion, wherein the tail end of the main absorbent segment connects to the back fixed portion by a connection portion, and wherein the tail end of the main absorbent segment is positioned at the back end of the back fixed portion.

5. The flexible sanitary pad fitted to the groin as claimed in claim 4, wherein the head end of the tilted tail portion connects to the back fixed portion, wherein the tail end of the main absorbent segment is aligned with a region between the back fixed portion and the tail end of the tilted tail portion, and wherein a length of the tilted tail portion is longer than the connection segment.

6. The flexible sanitary pad fitted to the groin as claimed in claim 1, wherein the suspended portion has a main absorbent segment, wherein a head end of the main absorbent segment is the front fixed portion, and a tail end of the main absorbent segment is the back fixed portion.

7. The flexible sanitary pad fitted to the groin as claimed in claim 1, wherein the suspended portion has a main absorbent segment, wherein a head end of the main absorbent segment connects to the back fixed portion by a connection portion, and the head end of the main absorbent segment is positioned at a front end of the front fixed portion, wherein a tail end of the main absorbent segment connects to the back fixed portion by another connection segment, and wherein the tail end of the main absorbent segment is positioned at a back end of the back fixed portion.

8. The flexible sanitary pad fitted to the groin as claimed in claim 7, wherein the main absorbent segment is tilted forward.

9. The flexible sanitary pad fitted to the groin as claimed in claim 4, wherein the fluid guide has a plurality of bended guide portion positioned between the head end of the main absorbent portion and the tail end of the main absorbent portion, and wherein a thickness of the fluid guide at the bended guide portion is smaller than a thickness of the fluid guide not at the bended guide portion.

10. The flexible sanitary pad fitted to the groin as claimed in claim 1, wherein the fluid guide has a front guide portion positioned at a front end of the front fixed portion, wherein a head end of the front guide portion is suspended without touching the absorbent pad, and wherein the head end of the front guide portion is tilted up.

11. The flexible sanitary pad fitted to the groin as claimed in claim 10, wherein the head end of the front guide portion is higher than the suspended portion.

12. The flexible sanitary pad fitted to the groin as claimed in claim 1, wherein the fluid guide has a front guide portion positioned at a front end of the front fixed portion, wherein the fluid guide has a plurality of bended guide portion positioned between a head end of the front guide portion and a tail end of the front guide portion, and wherein a thickness of the fluid guide at the bended guide portion is smaller than a thickness of the fluid guide not at the bended guide portion.

13. The flexible sanitary pad fitted to the groin as claimed in claim 1, wherein the fluid guide has a back portion connected to the absorbent portion and abutted against the tilted tail portion.

14. The flexible sanitary pad fitted to the groin as claimed in claim 1, wherein the fluid guide has an auxiliary block portion with a continuation segment connected to a abutting portion, wherein the continuation segment connects to the tail end of the tilted tail portion, wherein a head end of the abutting portion connects to the continuation segment, and wherein a tail end of the abutting portion is tilted up.
